(19) United States
(12) Patent Application Publication
(10) Pub. No.: US 2007/0288091 A1
(43) Pub. Date: Dec. 13, 2007

(54) INTERVERTEBRAL LORDATIC ADAPTER

(76) Inventors: Danny H. Braddock, Germantown, TN (US); Thomas A. Carls, Memphis, TN (US); Eric C. Lange, Collierville, TN (US)

Correspondence Address: HESLIN ROTHENBERG FARLEY & MESITI P.C., 5 COLUMBIA CIRCLE, ALBANY, NY, 12203, US

(21) Appl. No.: 11/421,119

(22) Filed: May 31, 2006

(51) Int. Cl. A61F 2/44

(52) U.S. Cl. 623/17.11

(57) ABSTRACT

An intervertebral implant adapter for the L5-S1 juncture comprises a wedge shaped body of biocompatible material having upper and lower surfaces with the upper surface having a slot which provides slidable interlocking engagement with a motion or fusion implant. The lower surface is provided with any convenient bone anchoring mechanism. The adapter mates with an upper implant which is implanted after the adapter so as to provide an implant which matches the lordotic angle but which, in its multiple piece form, is no bigger than the posterior disk height.

### TECHNICAL FIELD

The present invention relates generally to intervertebral implants and more particularly, to an adapter for use with spinal implants for use between the fifth lumbar vertebra (L5) and the sacrum (S1).

### BACKGROUND OF THE INVENTION

The human spine is a biomechanical structure with thirty-three vertebral members, and is responsible for protecting the spinal cord, nerve roots and internal organs of the thorax and abdomen. The spine also provides structural support for the body while permitting flexibility of motion. A significant portion of the population will experience back pain at some point in their lives resulting from a spinal condition. The pain may range from general discomfort to disabling pain that immobilizes the individual. Back pain may result from a trauma to the spine, be caused by the natural aging process, or may be the result of a degenerative disease or condition.

The intervertebral disc functions to stabilize the spine and to distribute forces between vertebral bodies. A normal disc includes a gelatinous nucleus pulposus, an annulus fibrosis and two vertebral end plates. The nucleus pulposus is surrounded and confined by the annulus fibrosis.

It is known that intervertebral discs are prone to injury and degeneration. For example, herniated discs are common, and typically occur when normal wear, or exceptional strain, causes a disc to rupture. Degenerative disc disease typically results from the normal aging process, in which the tissue gradually looses its natural water and elasticity, causing the degenerated disc to shrink and possibly to rupture. These conditions are often treated with the use of intervertebral implants. The L5-S1 juncture is one that is particularly prone to problems.

Thus, the region between the fifth lumbar vertebra and the sacrum (the L5-S1 region) is a region that is often treated with implant methods. However, the L5-S1 region is such that the distance between the bony structures increases greatly in the posterior to anterior direction. Certain implants, including those which are put in place from an anterior direction, are able to match the so-called L5-S1 lordotic angle only through the use of implants having a range of angled endplates; these implants are, however, single piece structures. Furthermore, the posterior introduction of a suitable implant for the L5-S1 region is very challenging. This is true whether or not the implant is a motion implant, which provides some spinal motion in the region, or a fusion implant in which relative motion of the spinal elements is substantially eliminated. A need therefore remains for an implant structure for use in the L5-S1 juncture.

### SUMMARY OF THE INVENTION

The shortcomings of the prior art are overcome and additional advantages are provided, in one aspect, through provision of an intervertebral implant adapter for the L5-S1 juncture comprises a wedge shaped body of biocompatible material having upper and lower surfaces with the upper surface having a slot which provides slidable interlocking engagement with a motion or fusion implant. The lower surface is provided with any convenient bone anchoring mechanism. The adapter mates with an upper implant which is implanted after the adapter so as to provide an implant which matches the lordotic angle but which, in its multiple piece form, is no bigger than the posterior disk height. The present adapter therefore enables posterior implantation in which the adapter is positioned first and anchored to the sacrum after which a motion implant or fusion implant is slidably interlocked with the adapter.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a side isometric view illustrating the L5-S1 lordotic angle, especially in comparison with the angle between other vertebrae;

FIG. 2 is a view similar to FIG. 1 except more particularly illustrating the placement of a motion implant and illustrating the lack of fit for such an implant at the L5-S1 location;

FIG. 3 is a view similar to FIG. 2 except that it shows the inclusion and placement of an adapter of the present invention with a motion implant;

FIG. 4 is the same view as FIG. 3 but from a partially frontal and partially side view;

FIG. 5 is a partially frontal and partially side isometric view illustrating positioning of a motion implant just prior to insertion and interlocking with the adapter of the present invention which is already in place;

FIG. 6 is an enlarged view similar to FIG. 5 but more particularly showing a motion implant partially engaged with the adapter;

FIG. 7 a partially frontal and partially side isometric view illustrating the use of the adapter of the present invention with a fusion implant;

FIG. 8 is an isometric view of an adapter in accordance with one embodiment of the present invention in which the lower surface of the adapter is provided with a keel for providing a bone anchoring surface;

FIG. 9 is an isometric view of an adapter in accordance with one embodiment of the present invention which is provided with a tab having an aperture for bone anchoring;

FIG. 10 is a view similar to FIGS. 8 and 9 particularly showing the inclusion of both a keel and tab structure for anchoring;

FIG. 11 is an isometric view of one embodiment of the adapter of the present invention illustrating the presence of slots or apertures for ingrowth of bone;

FIG. 12 is an isometric view of a motion implant usable in conjunction with the adapter of the present invention;

FIG. 13 is a view similar to FIG. 12 except showing the underside of a motion implant; and

FIG. 14 is an isometric view of a fusion implant usable in conjunction with the adapter of the present invention.

### BEST MODE FOR CARRYING OUT THE INVENTION

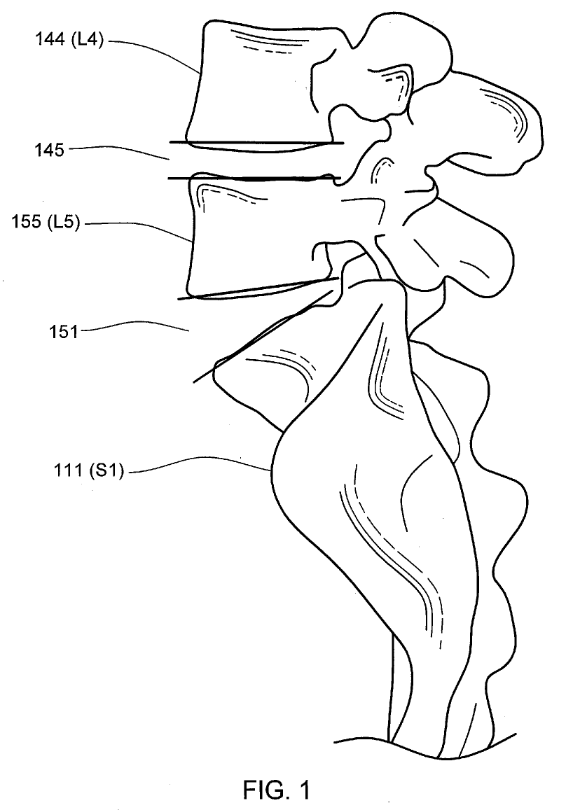

As indicated above, the present invention provides, in one aspect, an improved lordotic adapter. One of the problems solved by the present invention is illustrated in FIG. 1. Therein is shown a lower section of a spinal column particularly illustrating the fact that the intervertebral angle between L4 (the fourth lumbar vertebra, also designated by reference numeral 144) and L5 (the fifth lumbar vertebra, also designated by reference numeral 155) is essentially zero while on the other hand the intervertebral angle between L5 and S1 (the sacrum, also designated by reference numeral 111) is significant. Put another way, gap 151 between L5 and S1 (and more specifically, the top endplate of S1) increases in the posterior to anterior direction. The relatively large lordotic angle makes it difficult to provide implants for this region.

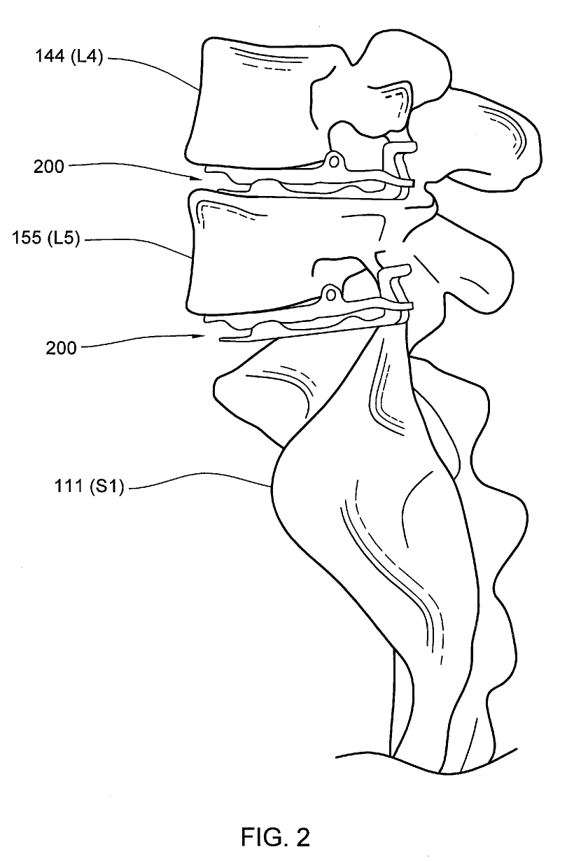

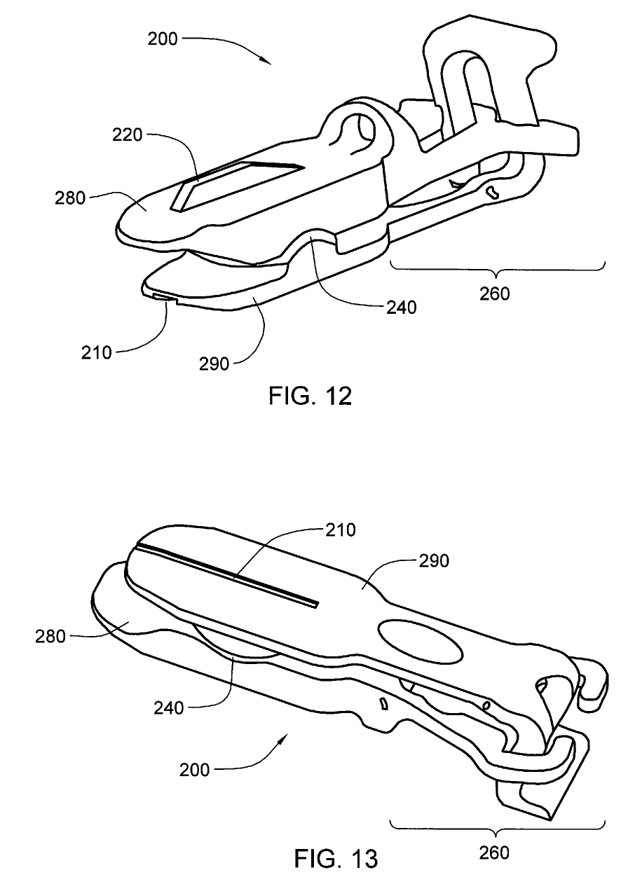

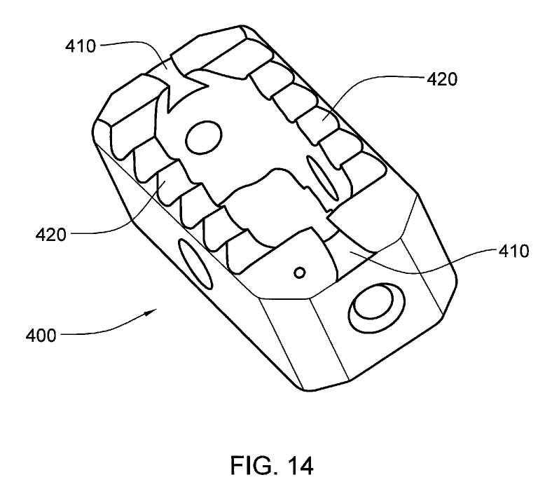

FIG. 2 illustrates the fact that implant 200, which is suitable for the L4 to L5 region, does not work for the L5 to S1 region. In particular, shown in FIG. 2 is motion implant 200 which is shown in more detail in FIGS. 12 and 13. However, it is noted that the adapter of the present invention works as well with fusion implant devices such as that shown in detail in FIG. 14.

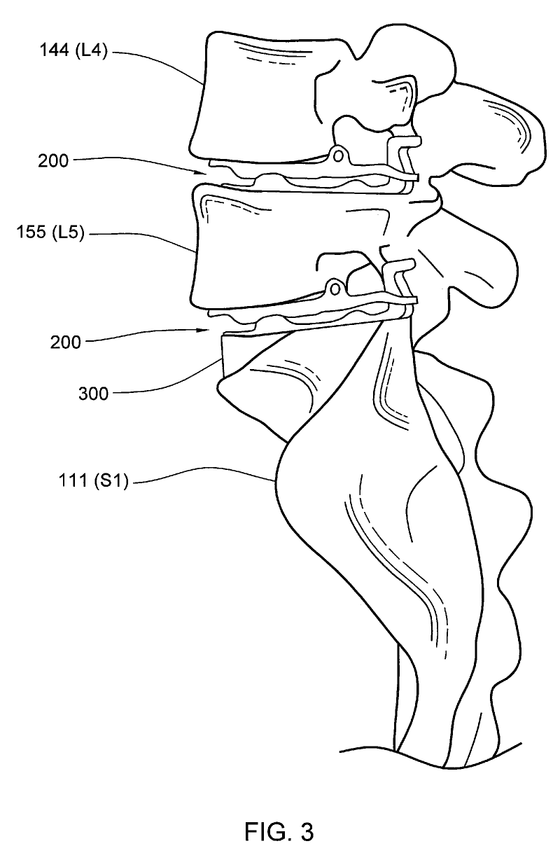

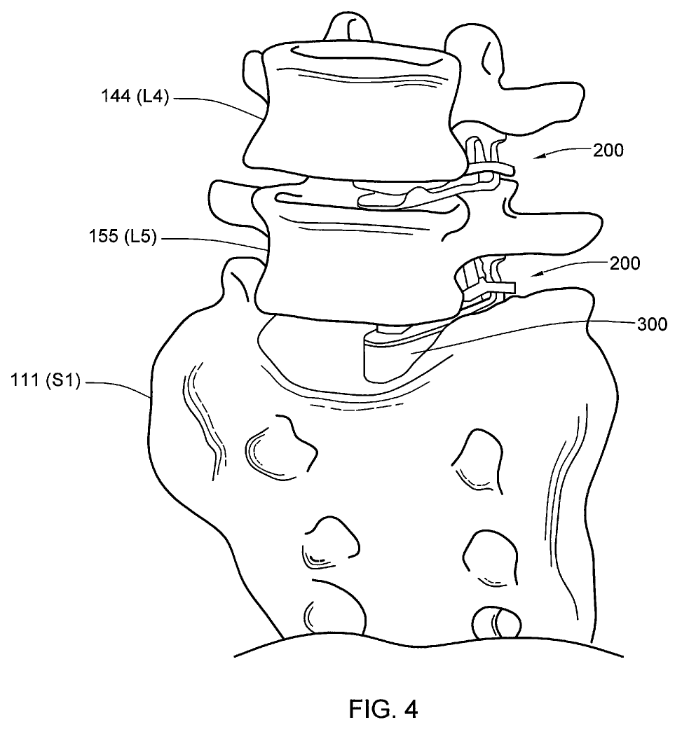

FIG. 3 illustrates the use and placement of the adapter of the present invention. Adapter 300 is disposed beneath motion implant 200 and above the S1 endplate to which it is affixed. FIG. 4 provides another view of the placement and positioning of adapter 300, particularly with respect to the sacrum. FIG. 4 is also useful for understanding that, when put in place from the posterior direction, there are usually two implants placed bilaterally. FIG. 4 illustrates the position of one of these implants and also illustrates not only the fact that there is room for a second implant but also shows where the second implant would be positioned laterally opposite to the first implant. When put into position from the anterior direction there is usually only a single implant device put into position. Such an implant usually spans the width of the disc space.

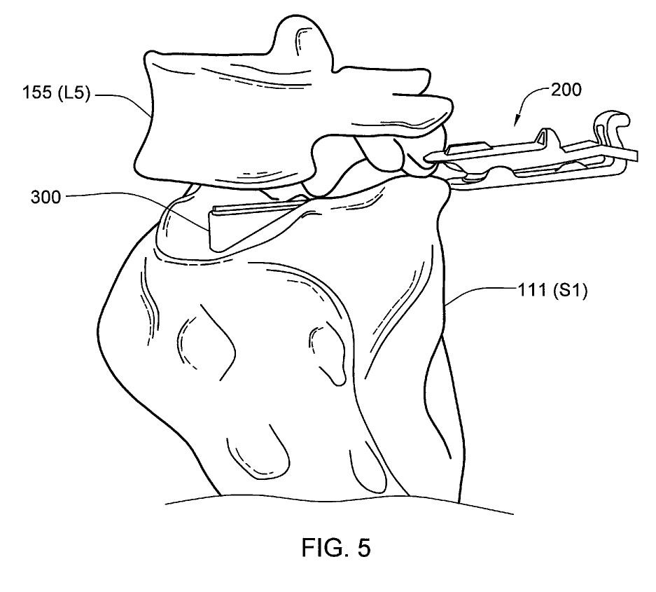

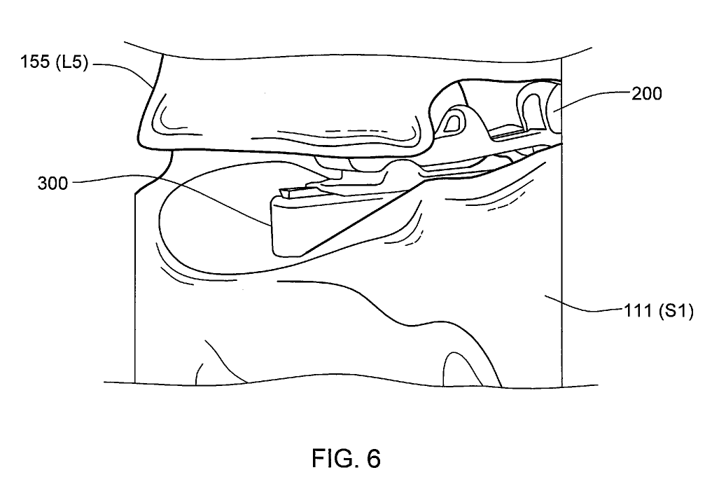

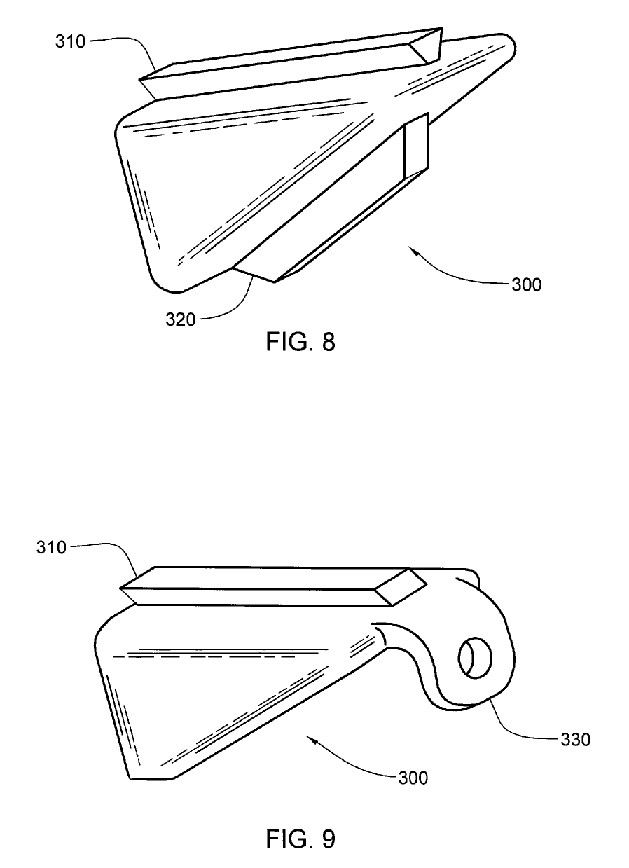

FIG. 5 provides an illustration of the order of placement of adapter 300 and motion implant 200. Adapter 300 is first affixed to the endplate of sacrum S1 (111). Affixation is by any convenient mechanism employed for this purpose. However, it is noted that adapter 300 is provided with a bone anchoring surface. Such a surface typically includes projections which extend downwardly from the lower surface of the adapter and/or includes apertures or other surface area enhancing features. Such features encourage growth of bone to improve adapter anchoring. After adapter 300 is put in place, implant 200 is slidably engaged with it. Details illustrating dovetail interlocking engagement are shown in FIGS. 8-14, both for motion implants 200 and fusion implants 400. FIG. 6 is similar to FIG. 5 except that it shows a more progressed stage of interlocking engagement.

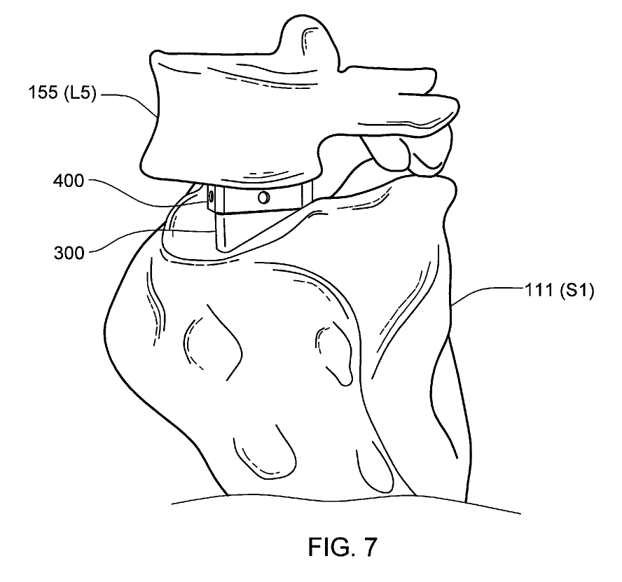

As noted above, adapter 300 is also employable with fusion implants. FIG. 7 illustrates the use of adapter 300 with fusion implant 400. Again, adapter 300 is put in place first after which implant 400 is slidably engaged with adapter 300. The interlocking engagement mechanisms usable for motion implants are also usable for fusion implants. As illustrated, the completed device now lies between L5 and S1, with fusion implant 400 being disposed above adapter 300 and interlocked therewith. An exemplary fusion implant is illustrated in more detail in FIG. 14 discussed more particularly below.

An exemplary adapter in accordance with the present invention is shown in detail in FIG. 8. In particular, adapter 300 is provided with keel shaped projection 320 which extends below the lower surface of adapter 300. This projection is placed within a correspondingly shaped depression surgically provided in the endplate of S1. It is also noted that adapter 300 is provided with dovetail shaped projection 310 extending from front to back along the upper surface of adapter 300. Adapter 300 itself is a wedge shaped body which comprises any convenient biocompatible material. It is also noted that adapter 300 may include materials which are not necessarily biocompatible but which are coated with one or more biocompatible materials. Biocompatible materials include titanium and bone.

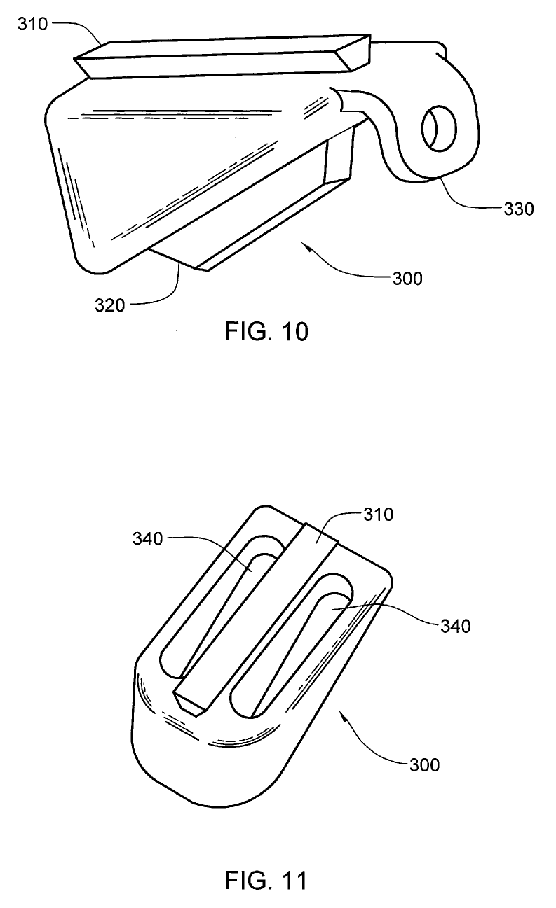

FIG. 9 illustrates a variation of adapter 300 in which, instead of keel 320, adapter 300 is provided with rear mounted tab 330 with an aperture through which a screw or bolt may be disposed for fastening to S1. FIG. 10 illustrates yet another variation possible with adapter 300, namely, one in which both keel 320 and tab 330 are employed. With respect to these features it is noted that, especially in embodiments in which tab 330 is absent, the lower surface of adapter 300 is a bone anchoring surface. If tab 330 is employed, it is not required that the lower surface of adapter 300 comprise a bone anchoring surface. Such surfaces may be provided with one or more downwardly projecting spikes or other protrusions for enhanced affixation to bone such as to the endplate of S1. Such surfaces may also include apertures, which tend to promote ingrowth of bone. These surfaces also include rippled surfaces such as those shown on the upper and lower surfaces of fusion implant 400 as seen in FIG. 14.

FIG. 11 illustrates another variation with respect to adapter 300. In particular, FIG. 11 illustrates the fact that adapter 300 may be provided with apertures 340 to promote the ingrowth of bone and thus to enhance fixation of the implant. In any event adapter 300 is still provided with interlocking protrusion 310 such as the dovetail structure shown herein.

FIGS. 12 through 13 illustrate the construction of a motion implant usable in conjunction with the present invention. Likewise, FIG. 14 illustrates the construction of a fusion implant usable in conjunction with the present invention. Except for the slidable interlocking engagement aspects, the detailed construction of either one of these implants is not relevant to the present invention. The present invention is fully usable with either fusion or motion implants.

With respect to FIG. 12, motion implant 200 comprises interlocking upper portion 280 and lower portion 290. Motion features are provided via pivot surfaces 240. Posterior elements 260, as shown, are provided for purposes not related to the present invention. Upper portion 280 of motion implant 200 is provided with keel shaped projection 220 which mates with a surgically created recess in L5. Most relevant for the present invention is the presence of mating dovetail slot 210. Dovetail slot 210 is more easily seen in FIG. 13 which depicts motion implant 200 from below.

FIG. 14 illustrates an exemplary fusion implant that may be employed along with adapter 300. Fusion implant 400 preferably includes ripples or ridges 420 which facilitate anchoring to bone. Fusion implant 400 is also shown in place in use with the present invention in FIG. 7. Again, most relevant for the present invention is the presence of mating dovetail slot 210. Dovetail slot 410 in FIG. 14, because of the relatively large central aperture in implant 400, is present in two locations as shown, but it functions in the same manner as described above for motion implant 200.

In the description above and the claims below, it is noted that references to upward or downward directions are relative. As used, these directions refer to implants as positioned in a individual standing upright with “upward” or “upper” referring to “toward the head” and “downward” or “lower” referring to “toward the feet.” Clearly, the adapter and the implants with which it is employed are usable in any spatial orientation.

Also, it is noted that in the description above, the adapter is shown with an upwardly extending protrusion along its upper surface for engagement with a slot on a motion implant or with a slot on a fusion implant. However, it is noted that the placement of the protrusion and the slot can be reversed, with the slot being on the adapter and the protrusion for slidable engagement being on the implant. The protrusion itself is of any convenient cross-section such as the dovetail cross-section shown. The upper part of the protrusion is wider than the lower portion to provide the slidable engagement. Clearly, too, the mating portions do not have to match exactly in cross-section. It is also noted that, while the figures show adapter 300 being disposed on the superior endplate of S1, it is also possible to dispose adapter 300 so as to engage the inferior endplate of L5. It is further noted that instead of an engagement which employs a slidable interlocking connection between an adapter and a corresponding implant, it is also possible to provide either one or both structures with pins that extend therefrom which match up with corresponding holes on the other piece. Snap connections between an adapter and its mating implant are also employable. These structures are more appropriate for anterior insertions as opposed to slidable interlocking engagements. Furthermore, the protrusions do not have to resemble pins per se. Any structure which protrudes from a mating surface between the adapter and an implant which is capable of insertion into corresponding recesses on the mating surface of the two devices is sufficient to provide an interlocking engagement which locks the two pieces together. Tapered protrusions and their corresponding recesses provide desirable structures for providing interlocking engagement between the two pieces. It does not matter which piece has protruding structures or which piece has corresponding recess structures. While it is thus seen that the engagement is not necessarily a slidable one, nonetheless, in slidable embodiments, even though the illustrations herein show a single slot and single mating structure, it should be appreciated that multiple slots may also be employed.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims. It is also noted that the present adapter and either a fusion or motion implant may be fixed in position relative to one another through the use of one or more screws. Screws for this purpose include set screws with a lock washer or screws with a spiral lock thread. When screws are used, the adapter and the implant are provided with overlapping parts so that the screws may be inserted in either a horizontal direction or at an angle to affix one piece to the other.

What is claimed is:

1. An intervertebral implant adapter comprising:
a wedge shaped body of biocompatible material having upper and lower surfaces:
said upper surface including at least one protrusion or recess which provides an interlocking engagement with an intervertebral implant; and with
said lower surface being a bone anchoring surface.

2. The adapter of claim 1 in which said at least one protrusion extends above said upper surface and provides slidable interlocking engagement with said intervertebral implant.

3. The adapter of claim 1 in which said recess extends below said upper surface and provides slidable interlocking engagement with said intervertebral implant.

4. The adapter of claim 1 in which said surfaces meet at a dihedral angle matching a lordotic angle between L5 and S1.

5. The adapter of claim 2 in which said protrusion is wider in a direction distal from said body.

6. The adapter of claim 2 in which said interlocking engagement is via a dovetail joint.

7. The adapter of claim 1 in which said bone anchoring surface comprises a downwardly extending keel.

8. The adapter of claim 1 in which said bone anchoring surface comprises a plurality of downwardly extending spikes.

9. The adapter of claim 1 in which said bone anchoring surface comprises a rippled surface.

10. The adapter of claim 1 in which said bone anchoring surface comprises at least one downwardly extending protrusion.

11. The adapter of claim 1 further including a tab at the narrow end thereof, said tab having an aperture therein.

12. The adapter of claim 2 in which said protrusion has a trapezoidal shaped cross-section.

13. The adapter of claim 2 in which said protrusion extends along the entire upper surface of said body.

14. The combination of the adapter of claim 1 and a motion implant having a matching surface which provides interlocking engagement with the upper surface of said adapter.

15. The combination of claim 14 in which said interlocking engagement is a slidable engagement.

16. The combination of the adapter of claim 1 and a fusion implant having a matching surface which provides interlocking engagement with the upper surface of said adapter.

17. The combination of claim 16 in which said interlocking engagement is a slidable engagement.

18. An intervertebral implant adapter comprising:
a wedge shaped body of biocompatible material having upper and lower surfaces:
said upper surface having a slot therein for slidable interlocking engagement with an intervertebral implant; and
said lower surface being a bone anchoring surface.

19. The combination of the adapter of claim 18 and a motion implant having a matching protrusion for slidable engagement with said slot.

20. The combination of the adapter of claim 18 and a fusion implant having a matching protrusion for slidable engagement with said slot.

21. An intervertebral implant adapter comprising:
a wedge shaped body of biocompatible material having upper and lower surfaces:
said upper surface having an upwardly extending protrusion for slidable interlocking engagement with an intervertebral implant; and with
said body being having a tab with an aperture at the narrow end of said wedge shaped body for anchoring said implant.

22. An intervertebral implant adapter comprising:
a wedge shaped body of biocompatible material having upper and lower surfaces;
said upper surface having at least one structure thereon for engaging a mating structure on an intervertebral implant; and with
said lower surface being a bone anchoring surface.

23. A method for inserting an intervertebral implant at the L5-S1 juncture, said method comprising the steps of:
first disposing a wedge shaped intervertebral adapter against an endplate of S1; and
then slidably engaging an implant between said adapter and L5.

24. The method of claim 23 in which said insertion is from the posterior.

25. The method of claim 23 in which said insertion is from the anterior.

26. The method of claim 23 in which said implant is a motion implant.

27. The method of claim 23 in which said implant is a fusion implant.

28. A method for inserting an intervertebral implant at the L5-S1 juncture, said method comprising the steps of:
first disposing a wedge shaped intervertebral adapter against an endplate of L5; and
then engaging an implant between said adapter and S1.

29. The method of claim 28 in which said insertion is from the posterior.

30. The method of claim 28 in which said insertion is from the anterior.

31. The method of claim 28 in which said implant is a motion implant.

32. The method of claim 28 in which said implant is a fusion implant.
